(19) United States
(12) Patent Application Publication
(10) Pub. No.: US 2013/0030246 A1
(43) Pub. Date: Jan. 31, 2013

(54) METHOD AND APPARATUS FOR LAPAROSCOPICALLY INDENTIFYING AND LOCATING STRUCTURES EMBEDDED IN FAT

(71) Applicant: Robert Paul Francis, Lewisville, TX (US)

(72) Inventor: Robert Paul Francis, Lewisville, TX (US)

(73) Assignee: RAYTHEON COMPANY, Waltham, MA (US)

(21) Appl. No.: 13/558,580

(22) Filed: Jul. 26, 2012

Related U.S. Application Data
(60) Provisional application No. 61/511,747, filed on Jul. 26, 2011.

(51) Int. Cl. A61B 1/313; A61B 1/06; A61B 1/05; A61B 1/04

(52) U.S. Cl. 600/109

(57) ABSTRACT

A scanning laser laparoscope for laparoscopically locating and identifying anatomical structures embedded within fat during microinvasive surgery. In one example, such a scanning laser laparoscope is achieved using a combination of a light source and a scanner located outside the body and configured to generate a pattern of light, an optics relay configured to relay the pattern of light toward and through a tissue section located inside the body, and a photodetector configured to receive the light pattern transmitted through the tissue section and to generate an image of the tissue section.

### RELATED APPLICATIONS

This application claims priority under 35 U.S.C. §119(e) to U.S. Provisional Application Ser. No. 61/511,747, entitled “METHOD AND APPARATUS FOR LAPAROSCOPICALLY IDENTIFYING AND LOCATING STRUCTURES EMBEDDED IN FAT,” filed on Jul. 26, 2011, which is hereby incorporated herein by reference in its entirety.

### BACKGROUND

The field of minimally invasive surgery has experienced dramatic growth in recent years. Many operations, including appendectomies, hysterectomies, and cholecystectomies, are currently performed by laparoscopy, a minimally invasive procedure associated with decreased risk of hemorrhage, reduced incidence of infection, and improved cosmetic results (i.e., reduced scarring). When performed safely, laparoscopic alternatives to traditional open surgeries can also reduce the costs of care by shortening hospital stays and recuperation times.

In traditional surgery, the patient's skin is cut along a length and retracted to reveal the target tissues of surgical interest. The surgeon has a large open field in which to visualize the important anatomical structures and operate. In minimally invasive surgery, by contrast, a portal is formed in the patient's skin and tools are inserted into the body cavity to permit visualization and dissection of the anatomy. Laparoscopic surgery is a type of minimally invasive surgery that provides direct visualization or access to a body cavity.

For laparoscopic surgery to be safe and effective, surgeons must have a clear view of the anatomical area and the target tissue to be treated. Often, however, important anatomical structures are embedded within the surrounding tissue, and are therefore difficult to localize and identify during laparoscopic surgery. For example, laparoscopic cholecystectomy is one of the most commonly performed operations in the United States, and a common complication of this operation is injury to the common bile duct, a branch of the biliary tree. The biliary tree, which comprises the cystic duct, the common hepatic duct, and the common bile duct (CBD), is often found embedded within surrounding fatty tissue. The cystic duct joins the common hepatic duct to form the common bile duct, which extends to an orifice leading into the small intestine. During open surgical cholecystectomy procedures, the surgeon is able to palpate the biliary tree and visualize the anatomical relationships between the various ducts prior to removing the gallbladder. During a laparoscopic cholecystectomy, however, the surgeon cannot so easily identify the anatomical relationships because the biliary tree is not tactilely accessible. Rather, the patient's anatomy is visualized two-dimensionally on a remote screen. Because structures within the biliary tree are often embedded in fat, surgeons must perform dissection around these structures blindly and may therefore inadvertently injure one of the structures.

Currently, the imaging modality commonly available for intraoperative CBD visualization is intraoperative cholangiography (IOC), an invasive technique in which a fluorescent dye is injected into the patient to visualize the ductal anatomy. IOC involves identification and cannulation of the cystic duct prior to obtaining a fluoroscopic image of the biliary tree. Although this provides the surgeon with a static map of the biliary tree, subsequent dissection remains blind and is only improved in that the surgeon has knowledge of the approximate location of important anatomical landmarks. In addition, the procedure is invasive, lengthens the surgical time, and adds complexity to the operation. Moreover, application of IOC to cholecystectomies has been shown to only modestly reduce the rate of injury to the CBD. Therefore, there exists a need to develop technology that would enable surgeons to better visualize the biliary tree prior to dissection of the gallbladder.

Another imaging modality that has been introduced for intraoperative visualization is optical coherence tomography (OCT) laparoscopy, which uses optical coherence technology to generate three dimensional images of the anatomy. The lengthy time requirements of OCT laparoscopy, however, limits the efficiency of this option for intraoperative imaging. In addition, OCT laparoscopy cannot image deeply into fat tissue to reveal structures embedded deeply within the fat tissue. Therefore, there exists a need to develop technology that would enable surgeons to better visualize the anatomical structures of interest prior to dissection through surrounding fat tissue.

### SUMMARY

Aspects and embodiments are directed to a scanning laser laparoscope system, which is inserted into the body of the patient, and that enables surgeons to visualize important anatomical structures and key anatomical relationships prior to or during dissection without the need for contrast agents and external imaging. The scanning laser laparoscope system enhances patient safety during minimally invasive procedures (such as laparoscopic procedures) and improves surgical outcomes would be significantly enhanced if there were imaging devices.

According to one embodiment, a scanning laser laparoscope for imaging tissue inside a body of a patient comprises a hollow housing including a proximal portion and a distal portion disposed opposite the proximal portion, the distal portion configure to enter the body, a light source disposed within the proximal portion, the light source configured to generate a collimated beam of light, a scanner disposed within the housing and configured to receive the collimated beam of light and direct the collimated beam of light along at least two axes to generate a light pattern, an optics relay disposed within the housing and configured to relay the light pattern from the scanner toward a tissue section inside the body, and a detection system including a photodetector positioned within the distal portion and configured to detect a transmission of the light pattern from the optics relay through the tissue section, the detection system configured to generate detection an image of the tissue section based on the transmission.

In one example, the detection system generates a three-dimensional image of the tissue section. In addition, the scanner may comprise at least one reflector configured to scan the collimated beam of light along the at least two axes. The scanner may further comprise a programmable position controller configured to control the scan of the collimated beam of light in a programmed scan sequence. In one example, the scanner is disposed within the distal portion. In another example, the scanner is disposed within the proximal portion.

In another example, the optics relay is configured to relay the light pattern through the housing from the proximal portion to the distal portion and toward the tissue section. In addition, the optics relay may comprise a first reflector and a second reflector, the first reflector disposed at the proximal portion and the second reflector disposed at the distal portion. Further, the first reflector may comprise a spherical or aspheric surface and the second reflector comprises a planar surface. In another example, the first reflector comprises a spherical or aspheric mirror and the second reflector comprises a spherical or aspheric mirror. In yet another example, the first reflector comprises a planar or aspherical minor and the second reflector comprises a planar or aspherical mirror.

In another example, the light source is configured to generate the collimated beam of light having a wavelength within a range from about 1400 nm to about 1500 nm, centered about 1450 nm. In addition, the light source may be configured to generate the collimated beam of light having a wavelength within a range from about 450 nm to about 1700 nm.

In another example, the photodetector includes an optical axis and is configured to detect the transmission of the light pattern through the tissue section along the optical axis at an angle θ with respect to the optics relay. In one example, the photodetector is configured to generate data representative of a shape and a position of the tissue section responsive to detection of the transmission, and wherein the detection system further includes a processor, and a communication circuit configured relay the data generated by the photodetector from the photodetector to the processor, wherein the processor is configured to generate the image of the tissue section based on the data.

In another example, the scanning laser laparoscope comprises an arm moveably connected to the distal portion of the housing such that the arm and the housing are configured to grasp the tissue section between them, wherein the photodetector is disposed on the arm. The photodetector may includes at least one Indium Gallium Arsenide (InGaAs) photodiode.

In another embodiment, a method of imaging tissue inside a body of a patient using a scanning laser laparoscope is disclosed, the scanning laser laparoscope having a housing including a proximal portion configured to remain outside the body and a distal portion configured to be inserted into the body, the housing including a light source, a scanner, and a photodetector. In one example, the method comprises inserting the distal portion into the body of the patient, positioning the distal portion adjacent to a tissue section inside the body of the patient, scanning, with the scanner, a first pattern of light emitted by the laser light source, relaying the first light pattern from the scanner toward the tissue section, detecting, with the photodetector, a second pattern of light transmitted through the tissue section, and generating an image of the tissue section based on the second pattern of light.

In another example, generating the image of the tissue section includes generating a three-dimensional image. In addition, the scanning laser laparoscope may further comprise an arm moveably connected to the distal portion of the housing. In this example, the method further comprises grasping the tissue section between the arm and the distal portion of the housing. In one example, the method further comprises scanning the light pattern along the at least two axes and controlling the scan of the first pattern of light in a programmed scan sequence.

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. In the figures, each identical or nearly identical component that is illustrated in various figures is represented by a like numeral. For purposes of clarity, not every component may be labeled in every figure. In the figures:

FIG. 1 is a schematic view of a scanning laser laparoscope system according to aspects of the present invention;

FIG. 2a is an illustration of the scanning laser laparoscope system of FIG. 1 inserted into a body cavity according to aspects of the present invention;

FIG. 2b is a schematic view of the distal portion of the scanning laser laparoscope system, according to aspects of the present invention;

FIG. 3a is a schematic view of the proximal portion of the scanning laser laparoscope system in a first position, according to aspects of the present invention;

FIG. 3b is schematic view of the reflector of the scanning laser laparoscope system, according to aspects of the present invention;

FIG. 3c is schematic view of the proximal portion of the scanning laser laparoscope system in a second position, according to aspects of the present invention;

FIG. 4a is a plan view of a photodetector of an exemplary scanning laser laparoscope system, according to aspects of the present invention;

FIG. 4b is a plan view of a window of an exemplary scanning laser laparoscope system, according to aspects of the present invention;

FIG. 5 is a ray trace showing light through tissue, according to aspects of the present invention; and

FIG. 6 shows a series of digital images obtained using a modeled scanning laser laparoscope system, according to aspects of the present invention.

### DETAILED DESCRIPTION

Aspects and embodiments are directed to a scanning laser laparoscope for laparoscopically locating and identifying anatomical structures embedded within fat during microinvasive surgery. According to one embodiment, such a scanning laser laparoscope is achieved using a combination of a light source and a scanner located outside the body and configured to generate a pattern of light, an optics relay configured to relay the pattern of light toward and through a tissue section located inside the body, and a photodetector configured to receive the light pattern transmitted through the tissue section and to generate an image of the tissue section.

As discussed in more detail below, when the pattern of light passes through the tissue section, different amounts of absorption of light in the anatomical structures and in the fat tissue results in the photodetector detecting different intensity of light corresponding different structures. The resulting image enables the user to visually observe if any anatomical structure of interest is embedded within the fat tissue prior to dissecting through the fat tissue. In some instances, embodiments of the present disclosure are configured to be part of an open surgical procedure or an endoscopic procedure.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

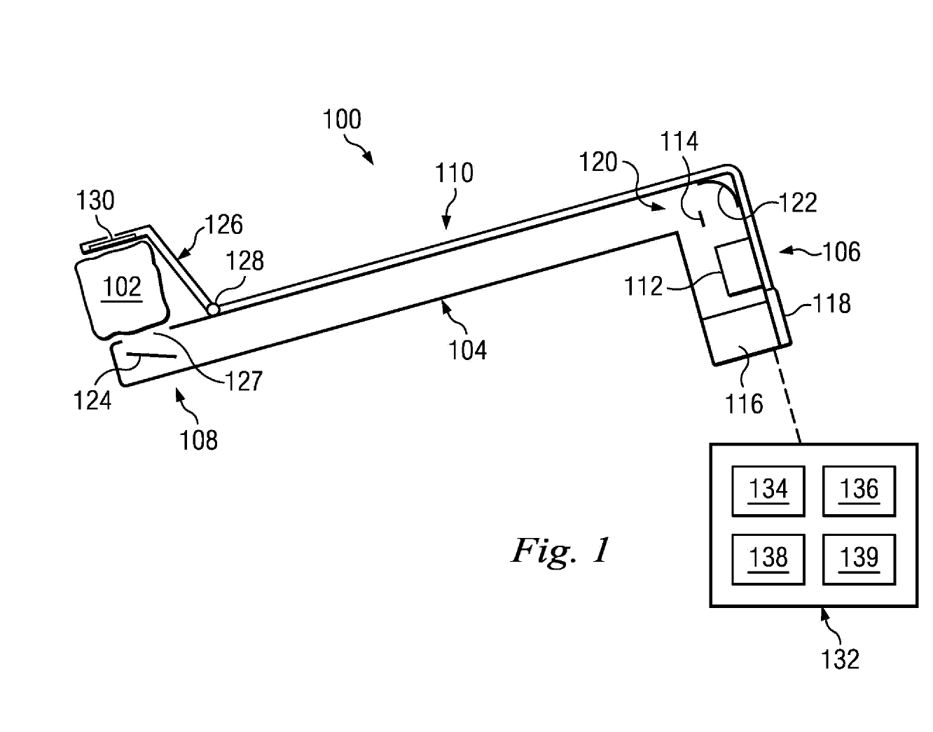

FIG. 1 illustrates a scanning laser laparoscope system 100 according to one embodiment of the present disclosure. The scanning laser laparoscope system 100 transmits a laser light pattern through a tissue section 102 located within a body cavity and generates, a image of the tissue section based on the transmitted pattern. The scanning laser laparoscope system 100 includes an elongate housing 104 with a proximal portion 106, a distal portion 108, and a channel portion 110 extending there between. The proximal portion 106 houses a light source 112, a scanner 114, and a control interface module 116. The proximal portion 106 may also include a handle 118.

The laser laparoscope system 100 further includes an optics relay 120, which includes a first reflector 122 and a second reflector 124. The first reflector 122 may be housed in the proximal portion 106 or a proximal end of the channel portion 110. The second reflector 124 may be housed in the distal portion 108 or a distal end of the channel portion 110. The system 100 further includes an arm 126 movable relative to the distal portion 108 of the elongate housing 104 by an actuating mechanism 128. A photodetector 130 is disposed on the arm 126. The distal portion 108 of the housing 104 includes a window 127. The light transmitted via the optics relay 120 through the window 127 may be emitted in the direction of the photodetector 130.

The scanning laser laparoscope system 100 also includes a control system 132, which comprises, in one example, a computer processor 134, a user interface 136, peripheral devices 138, and an image display system 139. The control system 132 may provide control commands to the control interface module 116 via a wired or wireless connection. As shown in the embodiment of FIG. 1, the control system 132 is physically separated from the elongate housing 104. However, in alternative embodiments, the control system 132 may be incorporated into the elongate housing 104. The components 134, 136, 138, 139 of the control system 132 may be housed in a single console or may be distributed throughout separate devices, including the elongate housing 104. The components of the control system 132 may communicate via wired or wireless connection with components in the housing 104, such as the light source 112, the actuating mechanism 128, or the photodetector 130.

In more detail, the channel portion 110 of the elongate housing 104 has a generally tubular construction and has a length sufficient to allow the proximal portion 106 to extend outside of the body while the distal portion 108 is positioned within the body cavity during use. As such, the overall length of the housing 104 may be of any length as is found in other laparoscopic or percutaneous devices. In some embodiments the cross-sectional shape of the channel portion 110 is circular. However, the channel portion 110 may have other cross-sectional shapes, for example square, rectangular, trapezoidal, triangular, or ellipsoidal, among other shapes. The cross-sectional dimension of the channel portion 110 may vary depending on the configuration of the scanning laser laparoscope 100, the size of the components of the optics relay 120, and other laparoscopic uses for the channel portion 110. In certain embodiments, the cross-sectional dimension of the channel portion ranges from 4 mm to 15 mm.

The handle 118 of the proximal portion 106 may include gripping features and other structures or mechanisms that may allow a user to grip and manipulate the system 100. The light source 112 may include any desired light having any desired wavelength. In one embodiment, the light source 112 may include a laser. In some embodiments, the light source 112 may include at least one laser diode or LED. In some embodiments, the light source 112 may include collimating optics (not shown) that collimate the generated light into a collimated light beam. In operation, the light source 112 may be modulated to generate a particular scan pattern either through direct modulation, by adjusting or interrupting the drive current, or through indirect modulation with an external modulator.

The control system 132 enables control of the laparoscope's various functions. In some embodiments, the control system 132 may comprise several individual user-controllable elements such as, for example, levers, buttons, turn-keys, dials, and knobs. Such user-controllable elements may be located on a separate control console or may be arranged on the proximal portion 106 of the elongate housing 104. In response to user manipulation of any of the user-controllable elements, the control system 132 may activate and/or control various actions of the laparoscope system 100 including the operation of the light source 112, the operation of the scanner 114, the operation of the optics relay 120, the movement of the arm 126 relative to the window 127, and the operation of the photodetector 130.

For example, a particular user-controllable interfacing element may be an ON/OFF button or switch which allows the laparoscope system 100 to be turned ON or OFF at the user's discretion. As another example, the characteristics and behavior of the light beam generated by the light source 112 may be controlled or adjusted by the control system 132. Such characteristics may include the wavelength, the frequency of modulation, and the pulse pattern. Some or all of these characteristics may be controlled based on specifications and inputs provided to the control system 132 by the user prior to and during the laparoscopic procedure. The user may also be able to define a range of wavelengths, scanning patterns, or various other parameters using the control system 132. For example, the control system 132 may allow the user to choose a particular wavelength emission from the light source 112 or direct the scanner 114 to scan a particular pattern or to scan a particular area over the tissue 102. The control system 132 may enable to user to guide the motion of the scanner 114, thereby guiding the direction at which the beams of the light source 112 are directed towards the tissue 102.

Within the control system 132, the processor 134 may be connected to the remote user interface 136 and the various peripheral devices 138. The processor 134 can receive input data from the remote user interface 136, the light source 112, the photodetector 130, the scanner 114, or any other components of the system 100. The processor 134 may be integrated within a computer and/or other types of processor-based devices suitable for a variety of scanning laser applications. The various peripheral devices 138 may enable or improve input/output functionality. Such peripheral devices 138 include, but are not necessarily limited to, graphical display screens, projectors, a CD-ROM drive, a flash drive, and a network connection. Such peripheral devices 138 may also be used for downloading software containing processor instructions to enable general operation of the scanning laser laparoscope system 100, and for downloading software implemented programs to perform operations to control, for example, the operation of any auxiliary devices attached to the system 100. The display system 139 may be a separate digital monitor, printer, or other digital output device.

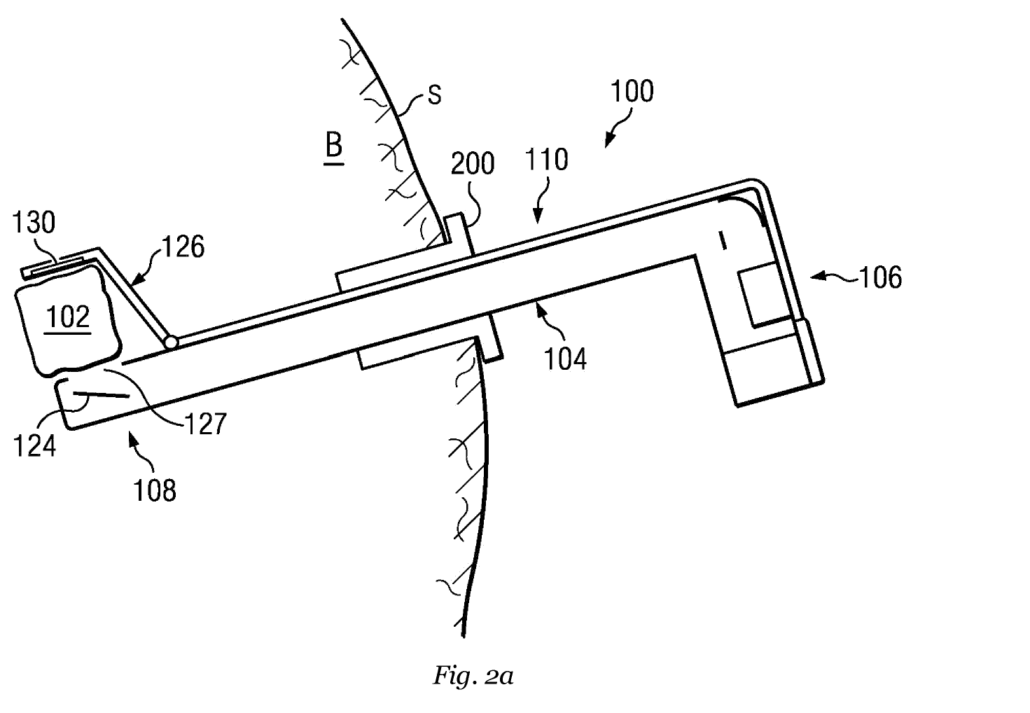

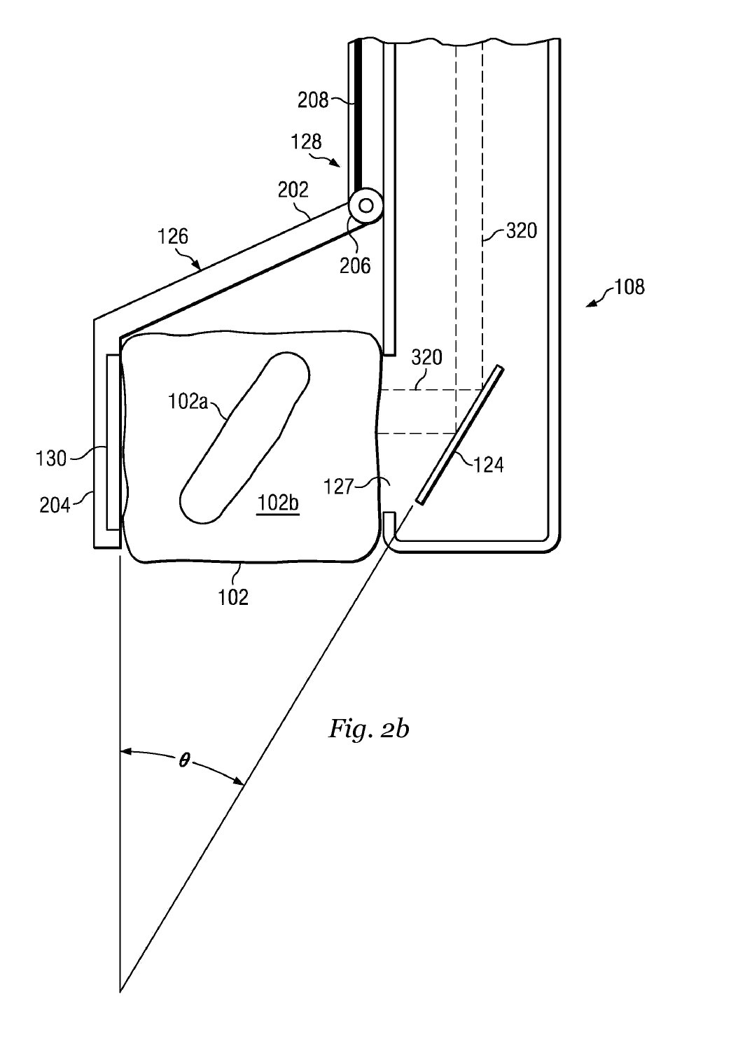

FIG. 2b illustrates the scanning laser laparoscope 100 inserted into a patient through the skin S into a body B. The laparoscope system 100 may be inserted into the body cavity of a patient by passing the distal portion 108 of the elongate housing 104 and at least a portion of the channel portion 110 through a lumen of a trocar 200, which is positioned at the skin surface S. The elongate housing 104 has a length sufficient to allow the proximal portion 106 to be outside of the body B while the distal portion 108 is positioned within the body during use. After the distal portion 108 of the elongate housing 104 is inserted into body B, the tissue 102 may be grasped between the arm 126 and the distal portion 108.

As depicted in greater detail in FIG. 2b, the arm 126 comprises a proximal segment 202 and a distal segment 204. The proximal segment 202 of the arm 126 is movable relative to the distal portion 108 of the elongate housing 104 such that the arm 126 and the elongate housing 104 can grasp the tissue 102 between the distal segment 204 and the distal portion 108 of the elongate housing. At least a portion of the arm 126 in proximity of the window 127 may have an irregular structure to enable improved traction with the tissue 102 to be grasped between the arm 126 and the distal portion 108. For example, the arm 126 may comprise a roughened or jagged surface.

The actuating mechanism 128 may be mechanically or electronically controlled to move the arm 126 relative to the housing 104. For example, the actuating mechanism 128 may include a movable connector 206 such as a swivel hinge, a spring-based connector, or a multi-axial connector. In the embodiment shown in FIG. 2b, the connector 206 is a hinge that enables the displacement of the arm 126 relative to the distal portion 108 of the elongate housing 104. In this embodiment, the distal segment 204 is generally parallel to the distal portion 108 of the elongate housing 104. In some embodiments, the proximal segment 202 and distal segment 204 of the arm 126 are configured to pivot relative to one another so that the arm 126 may lie flat against the elongate housing 104 when inserted into the body cavity through the trocar 200. Other configurations of the arm 126 may also be suitable for grasping tissue. For example, the segments of the arm 126 may be fixedly bent relative to each other. In another alternative, the arm 126 may have a single segment that extends at an acute angle relative to the distal portion of the housing 104.

Still referring to FIG. 2b, the actuating mechanism 128 includes an actuator 208 coupled to the connector 206 and extending toward the proximal portion 106 of the scanning laser laparoscope 100 such that the user may control the actuation of the arm 126. In some embodiments, the actuator 208 extends through the elongate housing 104. In other embodiments, the actuator 208 extends outside the elongate housing 104 of the scanning laser laparoscope system 100. In some embodiments, the actuator 208 connects to the control system 132 such that the user may control the movement of the arm 126 via one or more user controllable elements such as, for example, levers, buttons, turn-keys, dials, and knobs. The actuating mechanism 128 can comprise a variety of drive mechanisms, including, for example, electrical, mechanical, and/or electromechanical mechanisms.

Referring back to FIG. 2a, after the tissue 102 is securely grasped between the arm 126 and the distal portion 108 of the elongate housing 104, the light source 112 is activated. The light source 112 can emit light at a wavelength belonging within a variety of wavelengths, depending upon the desired imaging functionality of the scanning laser laparoscope system 100. In some embodiments, the light source 112 can emit monochrome or multi-color laser light. For example, where the desired functionality of the scanning laser laparoscope system 100 is to differentiate tissue structures having a higher water content than the surrounding adipose tissue (which has lower water content), the light source 112 is configured to emit light at wavelengths that correspond to the optical absorptions peaks associated with water in tissue. The light source 112 can therefore emit light within the short-wave infrared spectrum at a wavelength ranging from 1400 nm to 1500 nm. In one embodiment, a wavelength range centered around 1450 nm may be particularly suitable. The 1400 nm to 1500 nm wavelength range can be used to take advantage of the high absorption differential between the tissues containing more water (i.e. the CBD) and those containing less water (i.e. fat).

Although this short infrared spectrum is considered to be suitable for the water absorption differentiation application, the devices and methods described herein are not restricted to any specific type of laser or wavelength. For example, multiple wavelength lasers may be utilized to spectrally identify the absorbing structure embedded in the tissue 102, and to differentiate between absorbers which may be close to one another or overlapping spatially. In some embodiments, wavelengths ranging from 450 nm to 1700 nm can be utilized to differentiate between different anatomical structures having high water content.

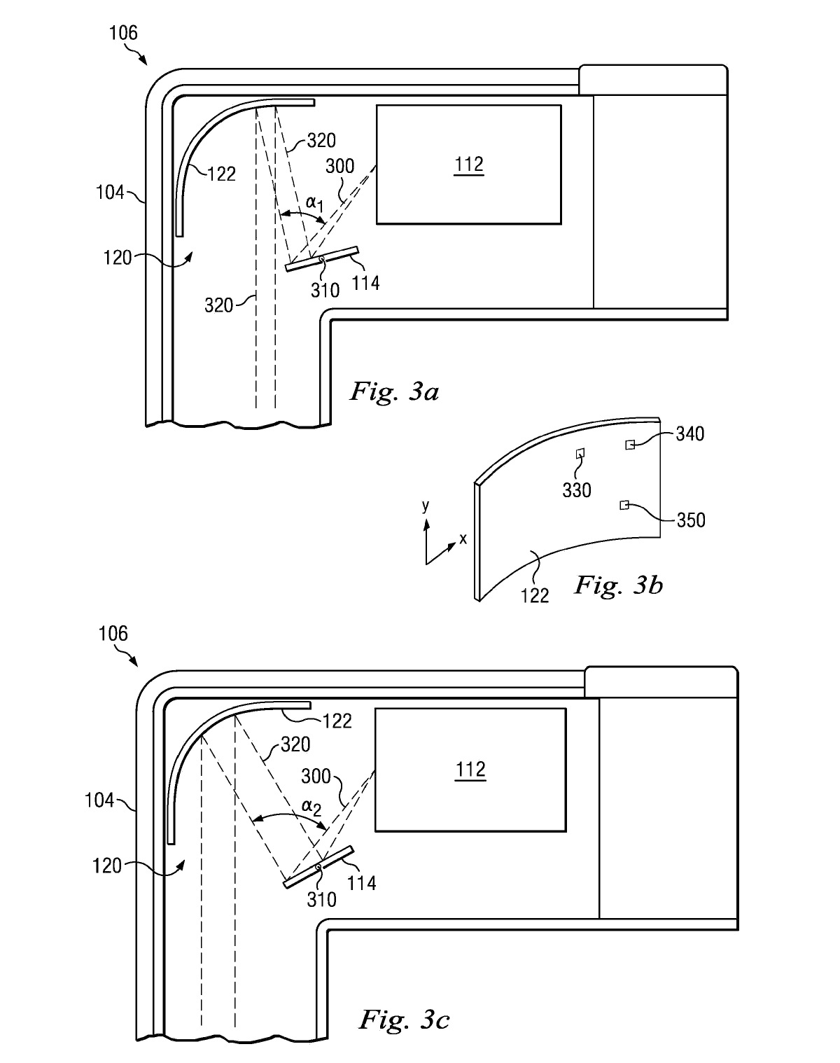

As shown in greater detail in FIGS. 3a, 3b, and 3c, the activated light source 112 emits a light beam 300 toward the scanner 114. The scanner 114 comprises an X, Y scanner reflector having a substantially flat reflecting surface. The scanner is shown to be located in the proximal end of the laparascope, but it may alternatively be located in the distal end of the laparoscope. The light beam 300 may have a width no greater than the smallest dimension of the scanner 114. When the light source 112 is activated, the emitted light beam 300 is incident upon the scanner 114. In this embodiment, the scanner 114 pivots about a central point 310 which allows the scanner to direct a reflected light beam 320 in at least two direction, for example the X and Y directions.

The reflecting surface of the scanner 114 may be driven by any of a variety of mechanisms to rotate about at least two axes either individually or simultaneously. For example, the scanner 114 may include micro-electromechanical systems (MEMS), which have a reflecting surface that may be driven electromagnetically, electrostatically, using a piezo crystal, or mechanically.

Scanning is accomplished by controlled movement of the scanner 114. In the embodiments shown in FIGS. 3a, 3b and 3c, the reflecting surface of the scanner 114 is rotated through two axes of rotation to direct the two axes scanned pattern of reflected light 320 into the optics relay 120. The two axes scanned pattern is shown in more detail in FIG. 3b. In one example, the reflected light beam 320 may be initially directed at a first location 330 on the first reflector 122 of the optics relay 120. By pivoting the scanner 114 about the pivot point 310, the reflected light beam 320 may be moved along the x-axis toward a second location 340. By further pivoting the scanner 114 about the pivot point 310, the reflected light beam 320 may be moved along the y-axis toward a third location 350. Thus, the reflecting surface of the scanner 114 angularly deflects the emitted light beam 300 in at least two axes. Each axis may be substantially orthogonal with each other to generate a scanned light pattern 330, 340, 350 along two substantially orthogonal axes. In some embodiments, the reflecting surface of the scanner 114 may be rotated through more than two axes of rotation to direct a multi-axial scanned pattern of light into the optics relay 120.

FIG. 3a depicts the emission and deflection of the emitted light beam 300 when the scanner 114 is in a first position, and FIG. 3c depicts the emission and deflection of the laser light beam 300 when the scanner 114 is in a second position. The first and second positions may correspond to the second and third locations 340 and 350 shown in FIG. 3b. The position of the scanner 114 determines an angle α at which the scanned light 320 is deflected into the first reflector 122 of the optics relays 120 and the particular location on which the scanned light 320 hits the first reflector 122. For example, the angle α₁ in FIG. 3a, where the scanner 114 is in a first position, is different than the angle α₂ in FIG. 3c, where the scanner 114 is in a second position.

In some embodiments, the scanner 114 may be programmed to scan a predetermined pattern. The control system 132 may communicate with the scanner 114 to provide control signals to the drive mechanisms of the scanner 114. The control system 132 controls the movement of the reflective surface of the scanner 114 so that the light beam 300 is redirected to provide a scan sequence and scanned pattern of light.

In one embodiment, the first reflector 122 comprises a powered reflector having a spherically or aspherically curved surface. In one embodiment, the reflecting surface of the first reflector 122 may be a mirror. However, other optical systems may be used, for example, the first reflector 122 may include one or more optical lenses. The first reflector 122 may be stationary within the proximal portion 106 of the housing 104. The scanner 114 may be located at the focal point of the first reflector 122. The first reflector 122 reflects the scanned light 320 toward the second reflector 124 in the distal portion 108. In this way, a pattern of parallel light beams, each in a different spatial location depending upon the orientation and angle of the scanner 114, is transmitted from the proximal portion 106 to the distal portion 108 of the elongate housing 104. In some embodiments, the first reflector 122 comprises a substantially planar reflector.

Referring again to FIG. 2b, the second reflector 124 in the optics relay 120 may be stationary within the distal portion 108 of the scanning laser laparoscope 100. In other embodiments, the orientation and position of the second reflector 124 in the optics relay relative to the elongate housing may be adjustable. The second reflector 124 functions to redirect the scanned pattern of light 320 through the window 127 of the distal portion 108 to become incident upon the tissue 102. A derivative pattern of light emerges from the tissue 102 and is detected by the photodetector 130. In one embodiment, the surface of the second reflector 124 may be a minor. However, other optical systems may be used, for example, the second reflector 124 may include one or more optical lenses. The second reflector 124 may transmit the received light 320 by tightening, maintaining, or increasing the overall projection field of the scanned pattern of light.

In the embodiment shown in FIG. 2b, the second reflector 124 comprises a substantially planar and flat surface positioned at an angle θ with relation to the photodetector 130. According to various embodiments, the surface geometry of the surface of the second reflector 124 can be configured to transmit various distributions of the scanned pattern of light. For example, in other embodiments, the second reflector 124 may comprise a spherical or aspheric surface. In some embodiments, the shape of the surface can be structured to converge the scanned pattern of light to increase the power density of the laser beams. In other embodiments, the shape of the surface can be adapted to compensate for non-uniform power distributions.

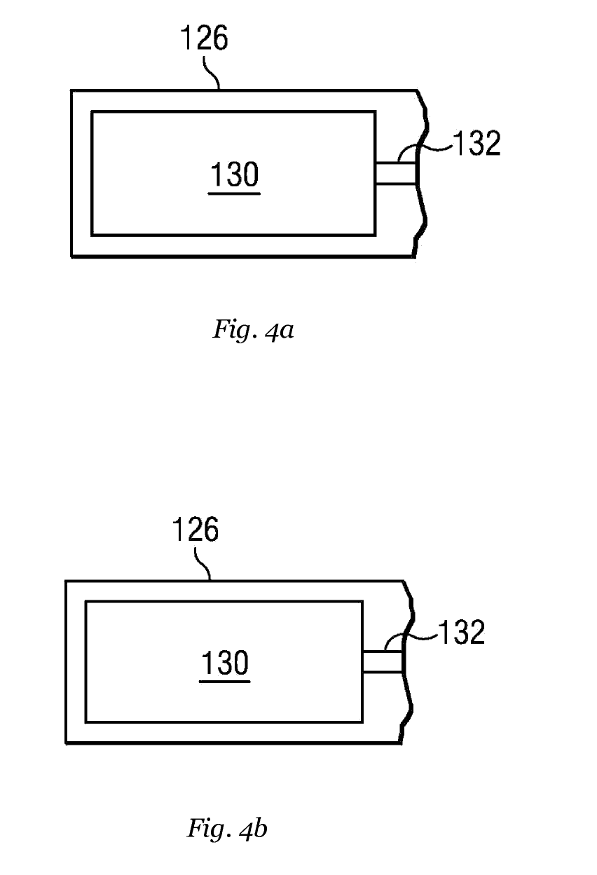

As shown in detail in FIG. 2b, the window 127 at the distal portion 108 of the elongate housing 104 is oriented to face the photodetector 130 in the arm 126. FIG. 4a shows a top view of the photodetector 130 included in the arm 126. The scanned pattern of light 320 reflects off the second reflector 124, travels through the window 127, and is directed through the tissue 102 to become incident upon the photodetector 130.

The window 127 in the elongate housing 104 is shaped and configured to allow the passage of the scanned pattern of light 320 reflecting from the second reflector 124. The window 127 enables the scanned pattern of light 320 to pass from the distal portion 108, toward the tissue 102. In some embodiments, the window 127 can be made from a transparent material. FIG. 4b shows one example of the window 127 included in the distal portion 108 of the elongate housing 104. In other embodiments, as pictured in FIG. 2b, the window 127 can constitute an empty space through which light 320 can pass.

In some embodiments, the distance between the arm 126 and the distal portion 108 (and thereby the distance between the photodetector 130 and the second reflector 124) can be controlled to control the focus/de-focus capabilities of the device 100, which also enables controlling the power density of the scanned pattern of light 320.

Referring again to FIG. 2b, the photodetector 130 is disposed approximately parallel to the window 127 and at the angle θ with respect to the second reflector 124. In some embodiments, the angle θ between the second reflector 124 and the photodetector 130 may be variable, such as when the photodetector 130 is at an oblique angle with respect to the window 127. The variance in angle θ may be an image compensation factor used to generate an image of the tissue 102.

The photodetector 130 may be a photo-sensitive or light sensitive device or an electronic circuit capable of generating a signal representative of the intensity of light detected. The photodetector 130 may be comprised of an array of individual single pixel photodetectors arranged so that each individual single pixel photodetector represents a picture or a pixel of a captured image. Each pixel may have a discrete position within the array. In some embodiments, each single pixel photodetector is an Indium Gallanium Arsenide (InGaAs) photodiode, a photodetector for the near infrared light range.

The photodetector 130 converts the incident light into an electrical output signal, which provides a measure of the incident energy of the transmitted pattern of light through the tissue 102. Thus, the photodetector 130 may comprise any appropriate light sensor device that converts incident light into an electrical signal and achieves the conversion with good linearity, good frequency range, and low distortion. The photodetector 130 can be a detector that detects light at one or more wavelengths.

The photodetector 130 may be coupled with a communication circuit of the control system 132. In some embodiments, the communication circuit comprises an electrical circuit that includes electrical and electronic components suitable for processing electrical signals of the scanning laser laparoscope system 100. The communication circuit may perform additional processing and communication with the other components of the control system 132 via a cable or wireless or some other communications link.

In some embodiments, the photodetector 130 may be coupled to a power circuit of the control system 132. The power circuit may be enclosed within a suitable enclosure, such as or in addition to the housing 104. The power circuit may be used to modulate the light source 112, to drive the motion of the scanner 114, and to gather electronic data received from the photodetector 200. In some embodiments, the power circuit and the communications circuit may comprise a single circuit.

Referring again to FIG. 2b, in some embodiments, when the scanned pattern of light 320 passes through the tissue 102, some structures within the tissue 102 may have a higher water content and can absorb more light than other structures. For example, structures such as the common bile duct 102a can absorb more light than the surrounding fat tissue 102b. The different amounts of absorption result in the photodetector 130 detecting lower intensity of light in the positions corresponding to the structures 102a, and higher intensity of light in the positions corresponding to the surrounding fat tissue 102b.

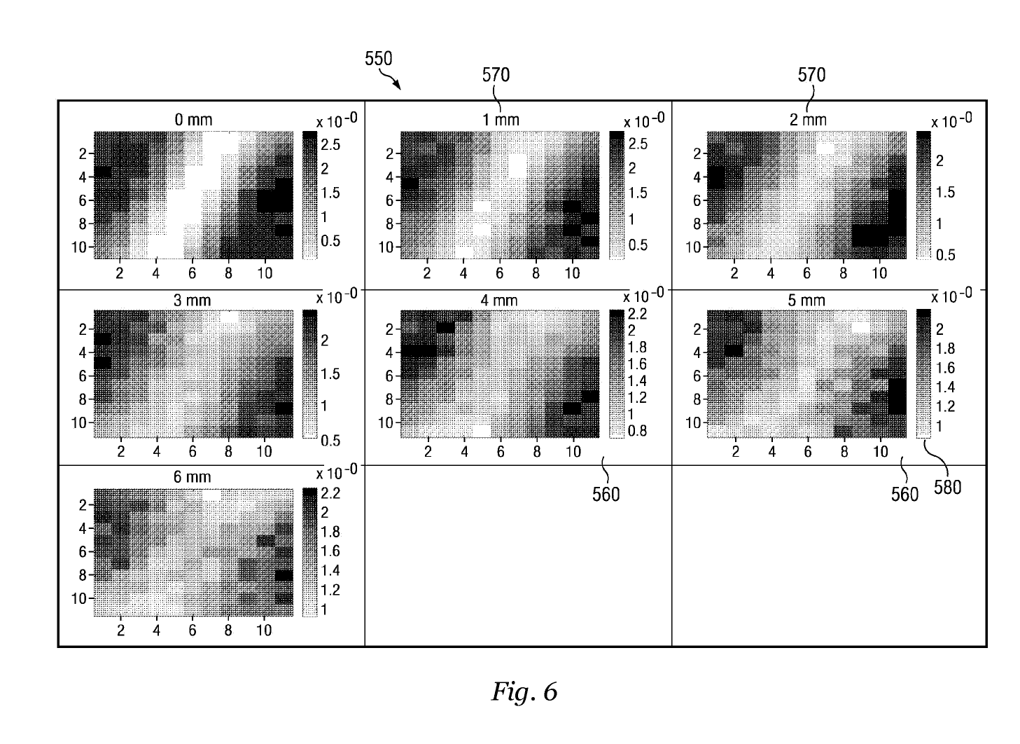

When the light is incident on the photodetector 130, an electrical signal corresponding to the intensity of the light is generated. The intensity sensed by each individual single pixel photodetector 130 is converted into electric signals that may be used to generate a digital image as will be described in greater detail in FIG. 6. Thus, the photodetector 130 responses at each individual position in the scan sequence captures an image of the structures 102a embedded within the fat tissue 102b of the tissue 102.

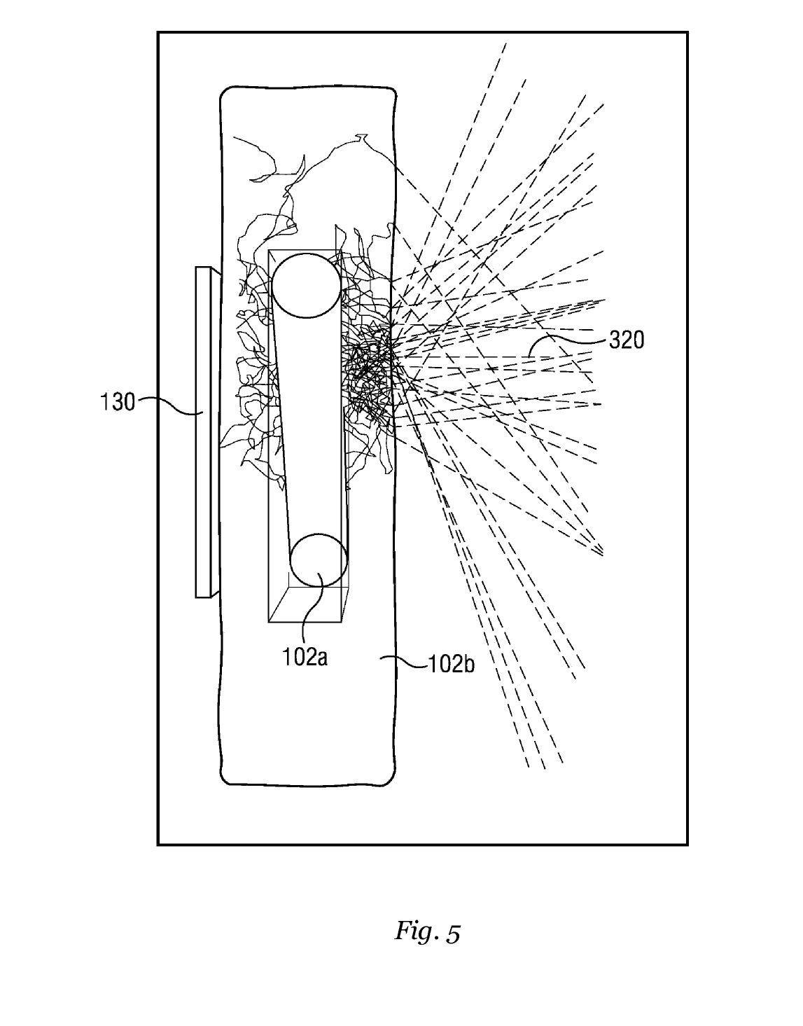

FIG. 5 shows one example of ray trace through tissue 102 having different absorption properties. As shown, the tissue 102 includes a rod structure 102a embedded within the fat tissue 102b or material with properties similar to fat or adipose tissue. For example, the structures 102a may represent gallbladder structures embedded within fat tissue 102a. In one example, the rod 102a has a 4 mm diameter and is embedded within a 10 mm thick block of fat tissue. The light 320 used to generate a scanned light pattern may include a laser beam having a 1437 nm wavelength.

The total response of the photodetector 130 is calculated for each X-Y location of the scan. The X-Y location may be moved as the scan proceeds, and an overall image may be created by stitching together the overall photodetector 130 response at each X-Y location. For example, if a scan has an 11×11 grid of different positions, the photodetector 130 response at each of the 121 X-Y locations is used to stitch the image of the target tissue. It is appreciated that the 11×11 grid is merely an example, and higher or lower resolution images may be suitable.

FIG. 6 shows a digital image 550 of the results obtained by scanning the tissue 102 with the structures 102a at seven different depths within the fat tissue 102b. The digital image 550 presents a visual grid 560 for each of the seven depths. Each visual grid 560 in FIG. 6 presents the “stitched-together” digital image of the overall photodetector 130 response at each of the 121 positions within the 11×11 grid. As shown, the 0 mm image shows the structure 102a at 0 mm from the surface of the fat tissue 102b, the 1 mm image shows the structure 102a at 1 mm from the surface of the fat tissue 102b, continually until the 6 mm image shows the structure 102a at 6 mm from the surface of the fat tissue 102b. The digital image 550 may be viewed with the image display system 139.

A depth marker 570 associated with each grid 560 indicates the distance between the embedded rod and the surface of the block closest to the window 127. An intensity legend 580 reflects the shade or color corresponding to the total light transmission (and thus total photodetector response) through the block. In this example, there is less contrast between the categories of light transmission in the legends 580 associated with increased depth of embedding.

As mentioned above, the image display system 139 and/or the processor 134 may be configured to receive the signals generated by the photodetector 130 that represent images of the transmitted light through the tissue 102. The image display system 139 together with the processor 134 may process the signals and display the image 550. The digital image 550 may be stored in the peripheral device 138, such as memory device.

The digital image 550 enables the user to visually observe if any anatomical structure of interest 102a is embedded within the fat tissue 102b prior to dissecting through the fat tissue. By using the scanning laser laparoscope to sequentially grasp and analyze various sections of fat tissue, the user may map out the locations of critical anatomical structures within a surgical area of interest. Other types of digital images may be developed using the signals received from the photodetector 130. For example, a composite image may be based upon sequentially scanned sections of tissue. A generated digital image may be static or dynamic. A generated digital image may be two dimensional or three dimensional. The use of a higher resolution scan pattern and/or corresponding photodetector may permit the formation of a higher resolution digital image.

The individual elements of the devices of the present disclosure may be fabricated from any convenient and appropriate material, provided that at least the distal portion 108 of the scanning laser laparoscope 100, the elements present at the distal portion 108, the arm 126, and the elements present at the arm 126 are fabricated from a biocompatible material. Biocompatible materials of interest include, but are not necessarily limited to, biocompatible polymers and biocompatible elastomers. Suitable biocompatible polymers include, but are not necessarily limited to, materials such as polyethelene, homopolymers and copolymers of vinyl acetate, polyvinylchlorides, homopolymers and copolymers of acrylates, polyvinylpyrrolidone, polyacrylonitrile butadiene, polyamides, cellulose acetate, polymethylpentene, polyurethanes, polycarbonates, fluoropolymers, polyesters, polyimides, polysulfones, polyisobutylene, polymethyisterene, silicone rubber, polyetheretherketone (PEEK), and other similar compounds known to those skilled in the art.

Suitable biocompatible elastomers include, but are not necessarily limited to, materials such as medical grade silicone rubbers, polyvinyl chloride elastomers, urethane-based elastomers, natural or synthetic rubbers, fluorenated polymers, and the like. In some embodiments, the laparoscope 100 may be fabricated at least in part from a radiopaque material. In other embodiments, the laparoscope may include radiopaque markers or some other imaging means to allow for visualization of the position of the laparoscope 100 within the body cavity. It should be understood that the possible biocompatible materials are included above for exemplary purposes and should not be construed as limiting.

Having described above several aspects of at least one embodiment, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What is claimed is:

1. A scanning laser laparoscope for imaging tissue inside a body of a patient, the scanning laser laparoscope comprising:
a hollow housing including a proximal portion and a distal portion disposed opposite the proximal portion, the distal portion configure to enter the body;
a light source disposed within the proximal portion, the light source configured to generate a collimated beam of light;
a scanner disposed within the housing and configured to receive the collimated beam of light and direct the collimated beam of light along at least two axes to generate a light pattern;
an optics relay disposed within the housing and configured to relay the light pattern from the scanner toward a tissue section inside the body; and
a detection system including a photodetector positioned within the distal portion and configured to detect a transmission of the light pattern from the optics relay through the tissue section, the detection system configured to generate an image of the tissue section based on the transmission.

2. The scanning laser laparoscope of claim 1, wherein the detection system generates a three-dimensional image of the tissue section.

3. The scanning laser laparoscope of claim 1, wherein the scanner comprises at least one reflector configured to scan the collimated beam of light along the at least two axes.

4. The scanning laser laparoscope of claim 3, wherein the scanner further comprises a programmable position controller configured to control the scan of the collimated beam of light in a programmed scan sequence.

5. The scanning laser laparoscope of claim 1, wherein the scanner is disposed within the distal portion.

6. The scanning laser laparoscope of claim 1, wherein the scanner is disposed within the proximal portion.

7. The scanning laser laparoscope of claim 6, wherein the optics relay is configured to relay the light pattern through the housing from the proximal portion to the distal portion and toward the tissue section.

8. The scanning laser laparoscope of claim 6, wherein the optics relay comprises a first reflector and a second reflector, the first reflector disposed at the proximal portion and the second reflector disposed at the distal portion.

9. The scanning laser laparoscope of claim 8, wherein the first reflector comprises a spherical or aspheric surface and the second reflector comprises a planar surface.

10. The scanning laser laparoscope of claim 8, wherein the first reflector comprises a spherical or aspheric surface and the second reflector comprises a spherical or aspheric surface.

11. The scanning laser laparoscope of claim 1, wherein the light source is configured to generate the collimated beam of light having a wavelength within a range from about 1400 nm to about 1500 nm, centered about 1450 nm.

12. The scanning laser laparoscope of claim 1, wherein the light source is configured to generate the collimated beam of light having a wavelength within a range from about 450 nm to about 1700 nm.

13. The scanning laser laparoscope of claim 1, wherein the photodetector includes an optical axis and is configured to detect the transmission of the light pattern through the tissue section along the optical axis at an angle θ with respect to the optics relay.

14. The scanning laser laparoscope of claim 1, wherein the photodetector is configured to generate data representative of a shape and a position of the tissue section responsive to detection of the transmission, and wherein the detection system further includes:
a processor; and
a communication circuit configured relay the data generated by the photodetector from the photodetector to the processor, wherein the processor is configured to generate the image of the tissue section based on the data.

15. The scanning laser laparoscope of claim 1, further comprising an arm moveably connected to the distal portion of the housing such that the arm and the housing are configured to grasp the tissue section between them, wherein the photodetector is disposed on the arm.

16. The scanning laser laparoscope of claim 1, wherein the photodetector includes at least one Indium Gallium Arsenide (InGaAs) photodiode.

17. A method of imaging tissue inside a body of a patient using a scanning laser laparoscope having a housing including a proximal portion configured to remain outside the body and a distal portion configured to be inserted into the body, the housing including a light source, a scanner, and a photodetector, the method comprising:
inserting the distal portion into the body of the patient;
positioning the distal portion adjacent to a tissue section inside the body of the patient;
scanning, with the scanner, a first pattern of light emitted by the light source;
relaying the first light pattern from the scanner toward the tissue section;
detecting, with the photodetector, a second pattern of light transmitted through the tissue section; and
generating an image of the tissue section based on the second pattern of light.

18. The method of claim 17, wherein generating the image of the tissue section includes generating a three-dimensional image.

19. The method of claim 17, further comprising scanning the light pattern along the at least two axes and controlling the scan of the first pattern of light in a programmed scan sequence.

20. The method of claim 17, wherein the scanning laser laparoscope further comprises an arm moveably connected to the distal portion of the housing, and wherein the method further comprises grasping the tissue section between the arm and the distal portion of the housing.
